(19) United States
(12) Patent Application Publication
(10) Pub. No.: US 2012/0130271 A1
(43) Pub. Date: May 24, 2012

(54) Self-Administered Hearing Test Kits, Systems and Methods

(76) Inventors: Robert H. Margolis, Arden Hills, MN (US); George Saly, Edina, MN (US); Mead Killion, Elk Grove Village, IL (US)

(21) Appl. No.: 12/952,589

(22) Filed: Nov. 23, 2010

(51) Int. Cl. A61B 5/00

(52) U.S. Cl. 600/559

(57) ABSTRACT

A kit for conducting a self-administered hearing test allows a test subject to generate calibrated pure tone stimuli during a hearing test without calibrating the testing system beforehand. Some hearing kits include a sound module and headphones configured to couple together and further couple with a processor-driven device, such as a computer. Some kits also include a computer-readable storage medium with executable instructions for programming a computer to perform the hearing test, including generating calibrated pure tone stimuli without calibration by the user. Methods and systems for conducting calibrated, self-administered hearing tests are also provided.

### STATEMENT REGARDING COPYRIGHT

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND

Testing of hearing sensitivity is usually performed by an audiologist using an audiometer to present a series of pure tone stimuli to a test subject at controlled frequencies and intensities. Hearing tests may be given in one or two parts, usually including an air-conduction test and/or a bone-conduction test. During air-conduction testing, earphones are worn by the subject and the sound travels through the air into the ear canal to stimulate the eardrum and then the auditory nerve. A tone at a particular frequency is presented to one ear, and its intensity is raised and lowered to find the lowest level (threshold) at which the person consistently responds. The person taking the test is instructed to give some type of response such as raising a finger or hand, pressing a button, pointing to the ear where the sound was received, or saying “yes” to indicate that the sound was heard. The subject's responses are recorded (e.g., manually or automatically), and the test results are typically plotted on an audiogram to illustrate the subject's hearing profile. Signals of different frequency may be consecutively presented to the same ear (e.g., commonly for at least six frequencies), and then the other ear can be tested in the same manner.

Audiometers are ordinarily calibrated, thus ensuring accurate and meaningful test results. For example, a calibration device is typically used to ensure that a pure tone stimulus emitted by an audiometer is within a desired tolerance relative to Reference Equivalent Sound Pressure Levels, such as those provided in the American and international standards for audiometers (e.g., ANSI S3.6-2004, ISO/DIS 389-8-2004). A number of different electroacoustic features of an audiometer can typically be calibrated, including sound pressure level, bone conduction force level (for bone-conduction testing), attenuator linearity, frequency, and harmonic distortion.

Accordingly, such hearing tests can be used to assess and interpret a subject's hearing thresholds and diagnose any hearing difficulties exhibited by the test subject. Of course these outcomes presume the availability of an accurately calibrated audiometer and a skilled and experienced audiologist who can conduct the hearing test and interpret its results. However, a person may desire to test his or her own hearing for a variety of reasons. For example, a person may find it inconvenient or expensive to schedule an appointment with a trained and licensed audiologist. In addition, a layman would in most cases not want to purchase a professionally calibrated audiometer, which can be challenging to operate and cost hundreds or thousands of dollars.

Self-administered hearing tests have been developed to address these types of situations. Unfortunately, current self-administered hearing tests exhibit one or more drawbacks. Some self-administered air-conduction hearing tests are incorporated into an audio compact disc (CD) or computer software that produces a series of tones. However, such self-administered hearing tests do not automatically produce calibrated pure tones and thus can only provide an estimated measurement of threshold levels. In addition, test users may employ a variety of headphones, each of which may produce different sound pressure levels for the same audio signals generated and output by the test.

For accurate measurements, a test user must often purchase a separate calibration device (e.g., a coupler and sound level meter) and then manually calibrate the output of the software program or audio CD. Aside from the disadvantage of the added cost, calibrating the tonal output can be difficult, especially for a layman who is not a trained audiologist. Furthermore, while some self-administered hearing tests attempt to measure and display threshold levels, the meaning and implications of such results can remain unclear to a test user unfamiliar with standard hearing profiles.

Accordingly, there is a need for further improvements in self-administered hearing tests.

### SUMMARY

According to one aspect of the invention, a kit for conducting a self-administered hearing test is provided. The kit includes a sound module having an input connector configured to couple the sound module to a computer and an output connector configured to couple the sound module to at least one headphone. The kit also includes at least one headphone having an input connector configured to couple the headphone to the output connector of the sound module. The kit also includes a computer-readable storage medium with executable instructions that cause a computer processor to perform steps in a self-administered hearing test. The steps in the self-administered hearing test include causing the sound module and the headphone to generate pure tone stimuli. The pure tone stimuli are calibrated to predetermined sound pressure levels without adjustment by a user administering the hearing test.

According to another aspect of the invention, a method for conducting a self-administered hearing test is provided. The method includes coupling a sound module to a computer and coupling headphones to the external sound module. The method also includes loading executable instructions onto the computer to perform steps in a self-administered hearing test. The method also includes conducting the self-administered hearing test with the computer to generate pure tone stimuli with the external sound module and the headphones. The pure tone stimuli are calibrated to predetermined sound pressure levels without a user adjusting the sound output levels.

According to another aspect of the invention, a system is provided for conducting a self-administered hearing test. The system includes a computer having a processor, at least one headphone, and a sound module coupled between the computer and the headphone. The sound module is external to, and removable from, the computer. The processor is programmed with executable instructions for conducting a hearing test. Upon receiving signals from the computer, the sound module and the at least one headphone produce pure tone stimuli calibrated to predetermined sound pressure levels without user adjustment.

Embodiments of the invention may provide one or more of the following features and/or advantages. Some embodiments include a portable, external sound module that can be easily and removably coupled to a computer using a communication protocol such as universal serial bus (USB). In some cases the predetermined sound pressure levels are selected relative to Reference Equivalent Sound Pressure Levels. For example, the levels may be specified in a standard issued by the American National Standards Institute. Some embodiments generate pure tone stimuli that are within an acceptable range or tolerance of the predetermined sound pressure levels. In some cases the pure tone stimuli are generated within about +/−3 dB of the predetermined sound pressure levels for frequencies below about 4.5 kHz, and within about +/−6 dB of the predetermined sound pressure levels for frequencies at or above about 4.5 kHz.

In some embodiments, the steps in the self-administered hearing test include receiving input signals from a user in response to the pure tone stimuli and identifying user threshold levels based on the input signals. In some cases the pure tone stimuli and user threshold levels are generated and identified, respectively, at one or more predetermined frequencies within a range of frequencies. Test result information based on the identified user threshold levels, and including a variety of information, may be communicated to a test subject in one or more formats. In some cases the test result information includes an interpretation (e.g., qualitative) or classification of the pattern of identified user threshold levels, and in some cases the test result information can include a user recommendation. Some embodiments communicate at least part of the test result information with an audio and/or video recording that may in some cases be played after setting a playback sound level based on the identified user threshold levels.

These and various other features and advantages will be apparent from a reading of the following detailed description.

### BRIEF DESCRIPTION OF THE DRAWINGS

The following drawings are illustrative of particular embodiments of the present invention and therefore do not limit the scope of the invention. The drawings are not to scale (unless so stated) and are intended for use in conjunction with the explanations in the following detailed description. Embodiments of the present invention will hereinafter be described in conjunction with the appended drawings, wherein like numerals denote like elements.

FIG. 1 is a depiction of a hearing test kit according to an embodiment of the invention.

FIG. 2 illustrates a graph of sound pressure levels at the eardrum of an earphone over a range of frequencies according to an embodiment of the invention.

FIG. 3 is a block diagram of a self-administered hearing test system according to an embodiment of the invention.

FIG. 4 is a flow diagram illustrating steps in a method for conducting a hearing test according to an embodiment of the invention.

FIG. 5 is a depiction of a form for displaying test results of a hearing test according to an embodiment of the invention.

### DETAILED DESCRIPTION OF THE PREFERRED EMBODIMENTS

The following detailed description is exemplary in nature and is not intended to limit the scope, applicability, or configuration of the invention in any way. Rather, the following description provides some practical illustrations for implementing exemplary embodiments of the present invention. Examples of constructions, materials, dimensions, and manufacturing processes are provided for selected elements, and all other elements employ that which is known to those of ordinary skill in the field of the invention. Those skilled in the art will recognize that many of the noted examples have a variety of suitable alternatives.

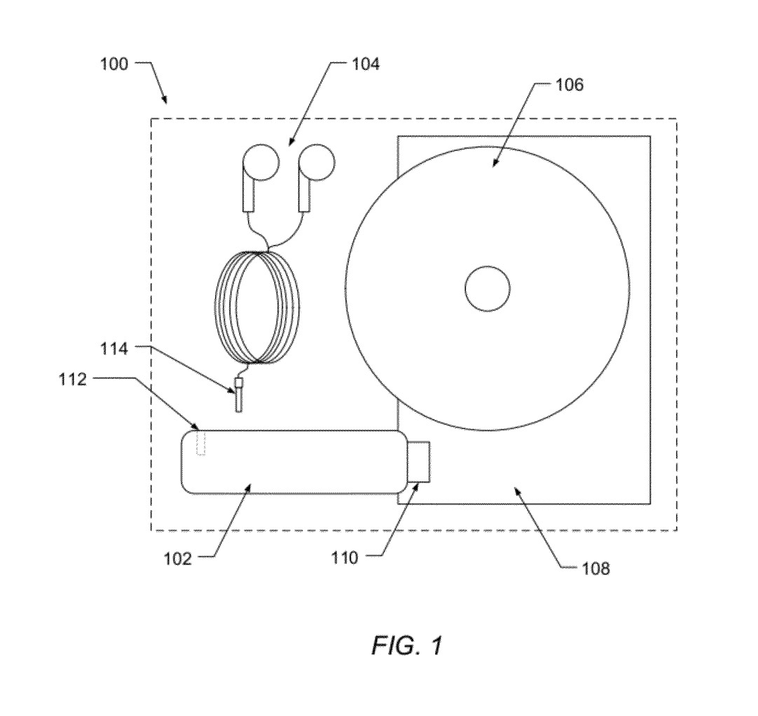

FIG. 1 is a depiction of a hearing test kit 100 according to an embodiment of the invention. Embodiments of the invention provide useful and convenient hearing test kits that allow a person to self-administer a hearing test using the kit and a computer. Self-administered hearing tests can be conducted at essentially any reasonably quiet location with a personal computer. Thus, the hearing test kit 100 is useful in a variety of settings. As just one example, FIG. 1 illustrates a home hearing test kit 100, which can be combined with a personal computer (e.g., laptop or desktop computer) to administer a hearing test in a person's home.

According to some embodiments, the hearing test kit 100 of FIG. 1 includes a number of components that when combined with a computer form a hearing test system that conducts a calibrated hearing test. Thus, the hearing test kit 100 provides a distinct advantage over currently available hearing test kits in that the assembled test system generates calibrated pure tone stimuli without the need to manually calibrate the hearing test system after set up. Accordingly, embodiments of the invention provide more accurate and reliable hearing test results than uncalibrated home hearing tests, and provide a calibrated sound output without the need for external calibrators and the expertise needed to calibrate the sound output of the headphones.

Returning to FIG. 1, in some embodiments the hearing test kit 100 includes a sound module 102 that can be coupled to a computer and one or more headphones 104 that couple to the sound module 102. The kit 100 also includes a computer-readable storage medium 106 storing a self-administered hearing test software program that, when installed on the computer, guides a user through a self-administered hearing test. The program generally produces calibrated, pure tone stimuli using the sound module 102 and the headphones 104, and records the user's responses to the stimuli to generate hearing test result information. In some cases, the hearing test kit 100 also includes product literature 108, an instruction manual, and/or other information that teaches a user about hearing loss, how to install and operate the hearing test software, etc.

According to some embodiments, the sound module 102 generally includes circuitry that receives signals from a computer and generates corresponding analog audio signals. For example, the sound module 102 can be a sound card for a computer. Such devices are well known in the art, and details of their construction and operation are omitted here. The sound module 102 is preferably an external, portable device that couples to an external port on a computer rather than being integral to or located within the computer. For convenience, the sound module 102 for the illustrated embodiment is alternately referred to herein as an “external sound module”, though it should be appreciated that the sound module is not restricted to an external configuration and may in some cases include an integral sound module located within the computer (e.g., a PCI bus sound card). In some cases the external sound module may couple to the computer via a universal serial bus (USB) connector 110. Other connectors and/or communication protocols may also be used (e.g., serial, parallel, 1394, Ethernet, wireless, etc.). Preferably, the external sound module 102 can be easily coupled and removed from the computer without opening the computer, as is generally necessary when installing an internal sound card connecting to the PCI bus. The external sound module 102 is preferably not within the computer itself, but may in some cases be partially or wholly received within an external computer slot (e.g., a PCMCIA slot).

The external sound module 102 also includes at least one audio output port or connector for coupling the sound module 102 with the headphones 104. For example, the module 102 may include a headphone jack 112 (e.g., RCA mini jack) that receives a plug 114 coupled to the headphones 104. Other known interfaces and connectors may also be used to couple the sound module to the headphones. According to some embodiments, the external sound module 102 is relatively small and portable, allowing it to be easily transported and coupled to computers in a variety of settings and locations as needed. In some cases, the sound module may resemble the size of a USB flash drive.

Returning to FIG. 1, the hearing test kit 100 includes a pair of headphones 104 that allow a user to hear the pure tone stimuli emitted by the external sound module 102. While a headphone pair is shown, it is contemplated that a single headphone may be employed in some embodiments. The headphones have an audio connector 114 (e.g., RCA mini plug) allowing them to be coupled to the external sound module 102. The term “headphone” is used herein to refer to all manner of audio speakers that are generally positioned on or near a user's head to transmit sound to the user's ear(s). For example, “headphones” may alternatively refer to earphones, earbuds, stereophones, headsets, and the like. The headphones 104 may cover the user's ears to varying degrees depending upon the embodiment and/or the background sound levels of the user's environment. For example, in some cases the headphones 104 may be circumaural headphones, supra-aural headphones, or canalphones.

The hearing test kit 100 in FIG. 1 also includes product literature 108, such as an instruction manual, hearing information, etc., and a computer-readable storage medium 106, such as a CD, containing hearing test software. In some cases the kit may include only minimal printed material or none at all. The hearing test software may be stored on a CD or any other suitable non-transitory computer-readable storage medium. Some examples include digital video discs (DVDs) and various forms of solid state memory, such as flash memory, although the invention is not limited to a particular format for the computer-readable storage medium. In some embodiments a user may install the hearing test software on a computer by separately downloading the software from a remote server over a network such as the Internet.

The hearing test software guides a test subject through a hearing test. In general, the software comprises executable instructions that cause the computer (i.e., the computer processor) to perform functions associated with the test. One example of a commercially-available hearing test software program is the Automated Method For Testing Auditory Sensitivity (AMTAS®) program available from Audiology Incorporated. During a typical self-administered hearing test, the computer processor will, among other things, instruct the external sound module 102 to generate a series of tones that are then transmitted to the headphones 104 and converted into pure tone stimuli that are audible to the test subject. For example, in some cases a series of tones over a frequency range of between about 500 Hz and 8 kHz are generated. In one embodiment, the series includes tones at these frequencies: 500 Hz, 1 kHz, 2 kHz, 4 kHz, and 8 kHz. As an initial step, the hearing test may instruct the computer processor to configure the volume settings of the external sound module 102 to intensity level(s) that have been predetermined according to the desired sound pressure levels to be emitted by the sound module and headphones. In some cases the software may further instruct the test subject (e.g., through a visual and/or audio instruction) to not manually change the volume settings of the external sound module, and/or that manually changing the volume settings may affect (e.g., invalidate) the calibration of the hearing test.

According to some embodiments, the emitted pure tone stimuli are calibrated to predetermined sound pressure levels without any action on the part of the person administering and taking the hearing test. For example, in some embodiments the kit's external sound module 102, the headphones 104, and the hearing test software cooperate together to achieve the desired calibration without a need for any manual adjustments by the user. The predetermined sound pressure levels can be any desired levels. In some cases the predetermined sound pressure levels are preferably a series of increasing sound pressure levels for each frequency measured relative to a Reference Equivalent Sound Pressure Level for the particular frequency (e.g., from 0 dB SPL to a 120 dB SPL). One example of Reference Equivalent Sound Pressure Levels that may be used are those provided in American and/or international standards for audiometers (e.g., ANSI S3.6-2004, ISO/DIS 389-8-2004).

The “pre-calibration” of the hearing test kit (e.g., before the kit is assembled, sold, and/or employed by a user) advantageously allows a test subject to conduct a calibrated, self-administered hearing test without needing to manually calibrate the output of the headphones as required in past self-administered hearing tests. Accordingly, some embodiments provide an economical and efficient, yet accurate, self-administered hearing test kit and testing method for non-experts. Further, a test subject/user does not need to purchase expensive and complex accessory items, such as a calibration coupler or a fully functional stand alone audiometer in order to conduct a calibrated, self-administered hearing test.

In some embodiments the pre-calibration can be achieved by selecting and/or providing the external sound module 102 and the pair of headphones 104 with minimum performance characteristics such as accuracy, precision and reliability. In some cases a particular external sound module and headphones may be selected based on one or more of these or other performance criteria. In some embodiments the headphones 104 may provide a desired absolute sensitivity, tolerance, and/or response over a range of frequencies. Table 1 provides a list of performance characteristics provided by one set of contemplated headphones.

TABLE 1

| Headphone | Value |
|---|---|
| Transducer | 8 mm Moving Coil |
| Transducer L/R Match | Within 2 dB at 1 kHz |
| Tolerance | +/−3 dB (400 Hz-4.5 KHz) |
| | +/−6 dB (4.5 KHz-9 KHz) |
| Sensitivity(@1 KHz) | 83 dB SPL for a 0.1 Vrms input into |
| | Zwislocki or G.R.A.S. ear simulator |
| Maximum output | 120 dB SPL |
| Maximum total | <2% @ 1 KHz at a nominal output |
| harmonic distortion(THD) | of 100 dB SPL (THD test conditions: |
| | 0.051 Vᵣₘₛ) |
| Maximum continuous input | 0.5 Vᵣₘₛ |
| Impedance: | 55-67 Ohms (nominal) |
| Noise isolation | 35 dB (3-flange ear tip deeply seated) |
| Operating Temperature | −10 to +50° |
| Storage Temperature | −20 to +60° |
| Testing Frequency Response | 400 Hz-9 kHz |

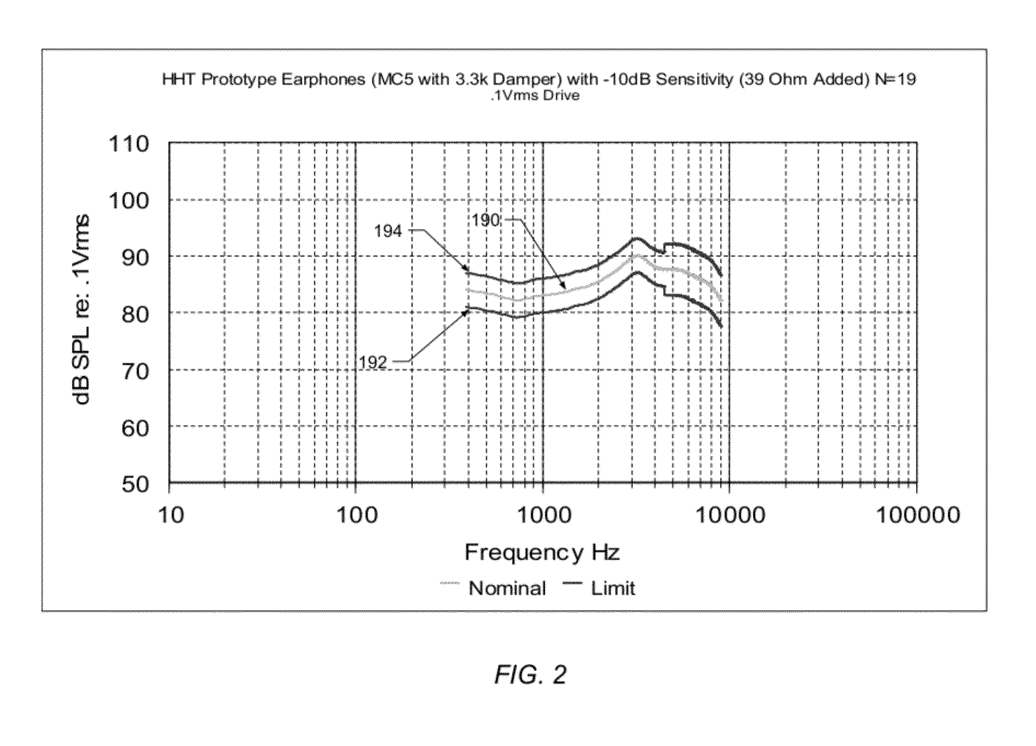

FIG. 2 illustrates the expected frequency response at the eardrum of one example of an earphone to a 0.1 Vᵣₘₛ input signal from the external sound module according to an embodiment of the invention. For example, an earphone provided with the characteristics listed in Table 1 exhibits the depicted nominal sound pressure levels 190 and minimum and maximum sound pressure levels 192, 194 over a range of testing frequencies (e.g., between 400 Hz and 9 kHz) according to an embodiment of the invention. Any suitable high-performance headphones exhibiting desired performance characteristics (e.g., suitably tight sensitivity tolerances) can be included with the kit 100. A number of potentially suitable headphones are available from Etymotic Research, Inc. For example, in some cases the headphones may be a version of mc5 earphones available from Etymotic Research, modified to enhance the sensitivity and tolerance of the headphones.

According to some embodiments, an improved frequency response in the headphones can be generated with the use of a passive electrical filter. For example, US Patent Application Publication 2007/0189569 describes an insert earphone with a high pass filter having resistance and capacitance values selected to correspond to the impedance of a moving coil driver. In addition, the frequency response of the headphones can be enhanced using a different number of damping elements, sound channels, auxiliary ducts, resonant ducts, auxiliary volumes, and/or electronic components, as described in the 2007/0189569 application, the entire content of which is hereby incorporated by reference. In one embodiment, the headphones include a modified version of the Etymotic Research mc5 earphones, including a 3,300 ohm damping resistor.

In some cases, the tolerance of the headphones' nominal sensitivity (e.g., in dB SPL) can be further narrowed by including an inline resistor within the headphones cable assembly matched to the headphones driver assembly. For example, prior to assembly, multiple headphone drivers may be sorted by sensitivity and matched with a cable assembly that has a resistance value suited to that particular driver. In some cases the headphone drivers may be sorted into low and high sensitivity drivers. In one example, modified mc5 earphones are sorted based on driver sensitivity. Earphones with low sensitivity drivers are then paired with a 27 ohm inline cable resistor and earphones with high sensitivity drivers are then paired with a 39 ohm inline cable resistor. Of course, a wide variety of resistance values may be provided depending upon the particular needs of a specific embodiment. In some cases the inline resistor is formed on a printed circuit board that is located within a cable splitter overmold.

In some embodiments the external sound module 102 may comprise the USB SoundWave 7.1 audio adapter (Part #: CE-500012-52) available from SIIG, Inc. Other possible external sound modules include the DA Series USB-to-Headset audio adapters available from Plantronics, Inc. Other similar known adapters capable of providing an audio driving signal that is adequate (e.g., within desirable tolerance levels) for the headphones 104 may also be used. Of course other known external sound modules and headphones meeting the minimum performance requirements may also be employed in some embodiments.

According to some embodiments, once a particular sound module and headphones are selected/provided for a kit, the hearing test software can be pre-calibrated to compensate for any irregular or undesired performance characteristics of the external sound module and/or the headphones. As just one example, the hearing test software instructions may be configured to signal the external sound module to output a slightly greater or lesser audio output signal to the headphones for a certain predetermined sound pressure level to compensate for undesired damping or amplifying effects of the external sound module and/or headphones.

In some embodiments the amount and kind of compensation/pre-calibration can be determined through a pre-calibration procedure prior to kit assembly (e.g., during kit development and/or manufacturing). For example, a headphone calibration coupler can be coupled to the selected headphones to monitor sound output during operation of the test and to determine deviation from desired reference levels. In some embodiments a coupler calibration procedure may be performed, such as a calibration procedure provided in ANSI/ASA S3.7-1995 (R2008). After determining the amount of correction needed to produce a calibrated output, the hearing test software can be programmed to adjust the input signals sent to the external sound module and the headphones in order to compensate for the inadequate performance characteristics of the external sound module 102 and/or the headphones 104. The software can then be finalized and included in each hearing test kit along with the selected sound module and headphones, providing a “pre-calibrated” hearing test kit.

In addition, in some embodiments the selected sound module and headphones preferably exhibit substantially the same performance characteristics (e.g., sound output is within a consistent tolerance range, different modules have a consistent maximum error threshold, etc.) throughout manufacturing lots. Thus, after pre-calibrating the hearing test, and a reference or prototype sound module and headphones combination, an entire lot of hearing kits will perform consistently without the need to pre-calibrate each kit. For example, a possible criteria for selecting a particular headphone is that the headphones are configured to receive an input signal from the external sound module and generate the pure tone stimuli within about +/−3 dB of the predetermined sound pressure levels for frequencies between about 400 Hz and about 4.5 kHz, and within about +/−6 dB of the predetermined sound pressure levels for frequencies between about 4.5 kHz and about 9 kHz. Consistency of performance across manufacturing lots may be determined experimentally and/or through documented product specifications.

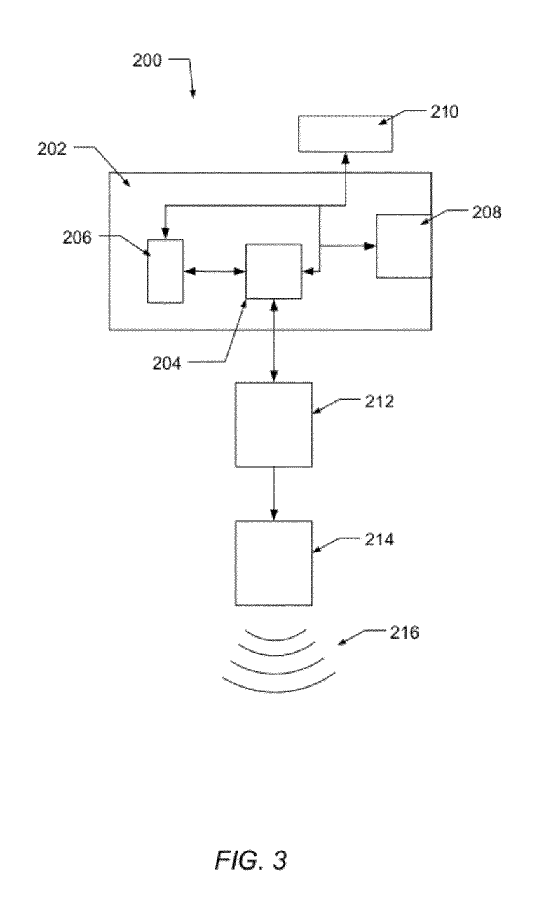

FIG. 3 shows a block diagram of a system 200 for conducting a self-administered hearing test according to some embodiments. In some embodiments, a home hearing test kit such as the one shown in FIG. 1 can be coupled with a computer 202 to form the system 200 shown in FIG. 3. Computers are well known in the art and for clarity, little detail is provided herein about the computer. Virtually any type of computer that 1) has an output connector for coupling the sound module, and 2) can be loaded with the hearing test software, can be incorporated into the testing system 200. In many cases, for example, a desktop or laptop personal computer may be used. However, other types of computers (e.g., processor-based devices such as personal digital assistants, mobile telephones, smartphones, digital media players, etc.) may also be employed depending upon the available processing power, memory capacity, etc. As shown in FIG. 3, the computer 202 has a processor 204 and a computer-readable storage medium 206 (e.g., read-only and/or read/write memory) that contains instructions that program the processor 204 to carry out specific functions. The computer 202 also has a data input means 208 (e.g., floppy/CD/DVD disk drive, USB port, network connection, etc.) that can receive the hearing test software and transfer it to the computer's storage medium 206. The computer 202 includes one or more user input and output devices 210 (display, keyboard, mouse, etc.) that allow a user to interact with the hearing testing program.

The external sound module 212 (e.g., an external sound card) is coupled to the computer 202 as described above and a single headphone 214 (or alternatively, a pair of headphones) is coupled to the external sound module 212. After loading and starting the hearing testing software on the computer 202, a user will hear one or more pure tone stimuli 216 from the headphone 214. The user can then respond to the tones and indicate through the computer input device 210 that he or she heard the tones. In some embodiments a user can open and install the hearing test software and then conduct a calibrated hearing test without adjusting the hearing test kit components.

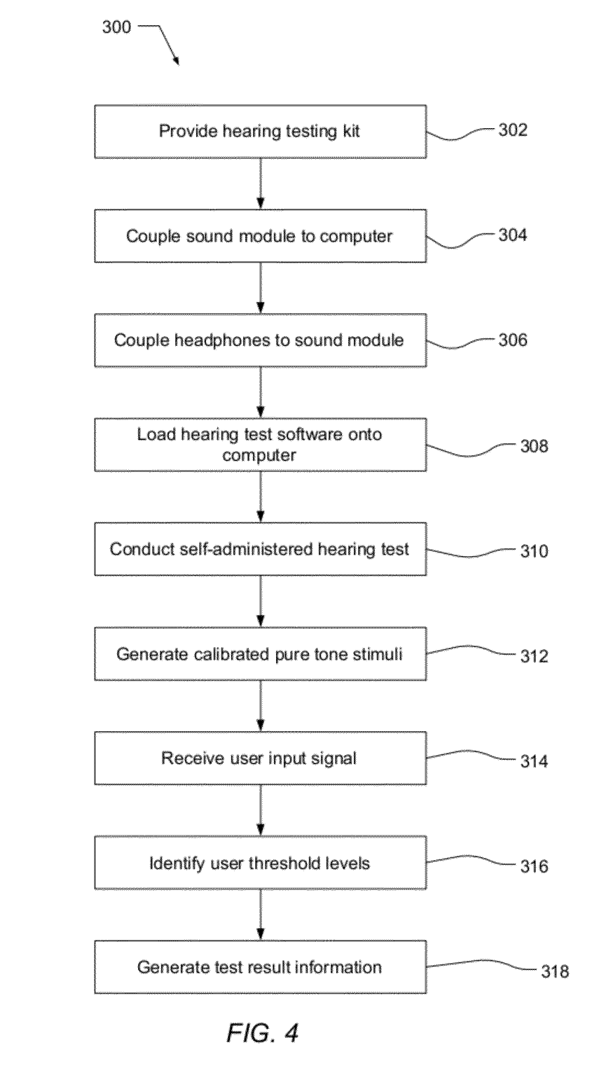

FIG. 4 is a flow diagram illustrating basic steps in a method 300 for conducting a self-administered hearing test according to some embodiments of the invention. The method 300 includes providing 302 a hearing test kit, such as one of the hearing test kits described herein. In some embodiments the hearing test kit consists essentially of and/or consists solely of an external sound module, headphones, and executable instructions that cause a computer processor to perform steps in a self-administered hearing test. The executable instructions may be stored on a locally present computer-readable storage medium or on a remote computer-readable storage medium available over a network connection. The hearing test kit does not include a calibrating mechanism, such as a headphone coupler. After providing the hearing test kit, the external sound module is coupled 304 to a computer and the headphones are coupled 306 to the external sound module. The executable instructions (e.g., the hearing test software program) are loaded 308 onto the computer through any conventional means, such as a removable computer-readable storage medium and/or by downloading the instructions over a network from a remote server. Then a self-administered hearing test can be conducted 310 in which the computer, sound module, and headphones generate 312 calibrated pure tone stimuli at one or more frequencies. The processor may then receive 314 an input signal from a test user/subject in response to each successive stimulus, indicating the person has heard the tone. The system may then identify 316 user threshold levels for the predetermined frequencies, and generate 318 test result information based on the identified user threshold levels.

The self-administered hearing test can be any test known in the art and threshold levels can be measured/identified according to any procedure known in the art. Preferably, the hearing test is one that conforms to the National Standard Methods for Manual Pure-Tone Audiometry (ANSI S3.21-1978) or automated methods that employ the principles embodied in that standard and have been validated against manual audiometry performed by an expert audiologist. For example, in some cases the hearing test may be administered using the AMTAS® program available from Audiology Incorporated, and described at least in part in U.S. Pat. No. 6,496,585, the content of which is herein incorporated by reference in its entirety. In many cases, such a test provides a software-controlled testing experience in which a set of acoustic stimuli are presented to the subject and the subject is asked to respond when he or she hears a stimulus. Each stimulus is typically an air-conducted pure tone stimulus. The stimuli are presented to the subject at different frequencies and intensities. Air-conduction transducers (e.g., headphones) are placed over or in the user's ears and then stimuli are delivered through the transducers to each ear. In some cases air-conducted masking noise is presented to the non-test ear during testing to insure that the non-test ear is not hearing the stimuli. The processor-driven program receives from the subject responses to the stimuli (e.g., through the computer input devices). Based on the responses received, the user's threshold levels at various sound frequencies are identified. In some embodiments the hearing test software generates test result information that often includes a standard clinical pure tone audiogram comprised of thresholds for tonal stimuli varying in frequency between 250 and 8000 Hz presented by air-conduction.

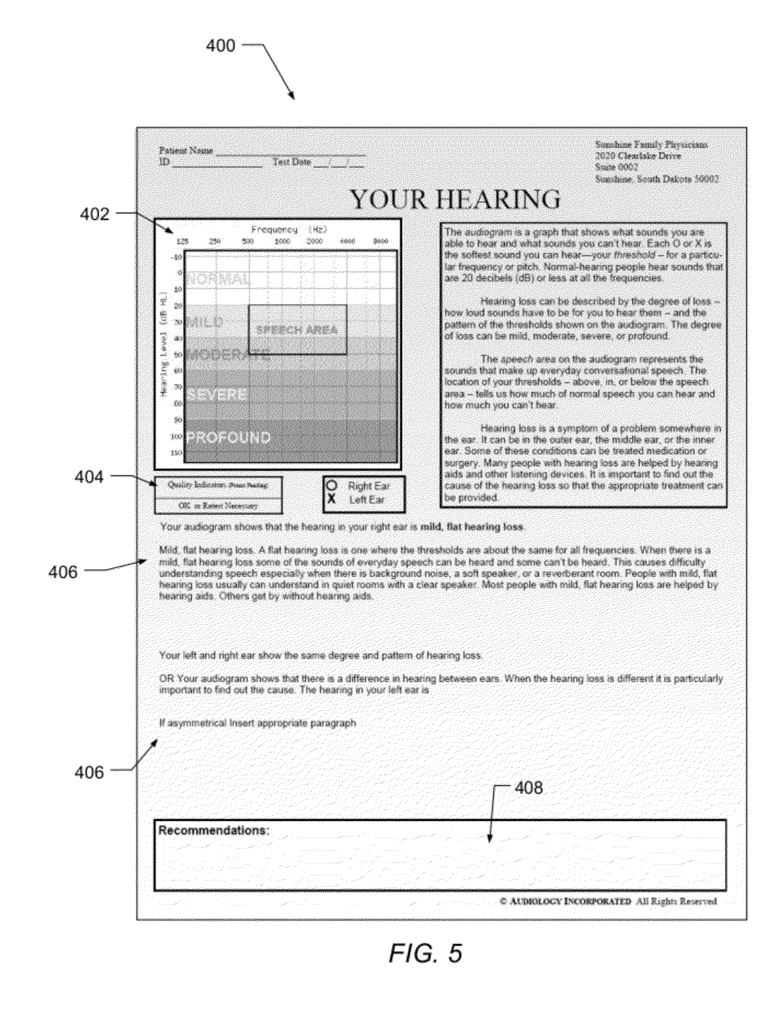

The hearing test software preferably generates test result information based on the identified user threshold values. Turning to FIG. 5, in some embodiments the test result information includes an automatically-generated report 400 (e.g., in electronic or paper form). In some cases the threshold values for both the left and right ears are plotted on an audiogram 402 for a visual depiction of the user's hearing profiles.

According to some embodiments, at the same time the user's threshold levels are being identified, the processor-driven, self-administered hearing test monitors and/or measures one or more quality indicators 404. The quality indicators correlate to probable test accuracy for a specific test. The quality indicators employed may differ depending on the specific test method used. Some quality indicators that may be measured include a time per observation interval, an average number of trials for a threshold, a false alarm rate, and/or a quality check fail rate. It should be appreciated that the invention is not limited to the measurement of any particular quality indicator. Several possible quality indicators and methods for assessing them are described in U.S. Pat. No. 6,496,585 and U.S. Pat. No. 7,704,216, the contents each of which are herein incorporated by reference in their respective entireties.

At the end of the test, the quality indicators 404 may be indicated in the report 400. In some cases, the test result information may only indicate that the quality indicators are adequate (e.g., the test was sufficiently accurate) or that a retest is necessary.

In addition to providing quantitative threshold data, some embodiments of the hearing test software preferably also convey qualitative hearing test result information. Referring again to FIG. 5, in some embodiments the report 400 includes a qualitative interpretation 406 of the identified user threshold values that explains to the user the nature and severity of the hearing loss (if any). The interpretive information 406 can include any useful information corresponding to the identified user threshold levels. In some cases one or more standard interpretive statements may be identified based on the threshold level and then inserted into the report 400.

According to some embodiments, the interpretive information 406 may include a classification for the user's hearing results and/or a statement of the likely communicative difficulties resulting from the identified hearing loss. For example, after determining the threshold levels for one or both ears, the hearing test software may then proceed to classify the identified levels based on a classification scheme. Several classification schemes are possible, and the invention is not limited to any particular classification scheme. In some embodiments the classification may proceed according to one or more schemes as outlined in U.S. Patent Application Publication No. 2008/0221719, the entire contents of which is herein incorporated by reference.

Further the hearing test software may in some cases provide recommendations 408 to the test subject based on the identified threshold values. For example, the hearing test software may recommend that the user follow up the self-administered test with a clinical appointment to determine the nature and severity of any hearing loss.

Referring to FIG. 5, in some embodiments test result information (e.g., an interpretation of the hearing test results) may be presented in text format in the automatically-generated report 400. In some embodiments, test result information may additionally, or instead, be presented in an audio and/or video format. For example, upon completion of the hearing test a movie file can be played that explains the user's hearing test results (e.g., audiogram) to the user. In some embodiments the hearing test software may include a number of prerecorded audio and/or video files, each of which correspond to particular test result information (e.g., interpretations, recommendations, classifications, etc.). In some embodiments the sound level for playback of the audio and/or video recording is determined and set based on the identified user threshold levels to ensure that the file(s) are presented at a comfortable and intelligible level for the hearing test subject.

Thus, embodiments of the invention are disclosed. Although the present invention has been described in considerable detail with reference to certain disclosed embodiments, the disclosed embodiments are presented for purposes of illustration and not limitation and other embodiments of the invention are possible. One skilled in the art will appreciate that various changes, adaptations, and modifications may be made without departing from the spirit of the invention.

What is claimed is:

1. A kit for conducting a self-administered hearing test, comprising:
a sound module having an input connector configured to couple the sound module to a computer and an output connector configured to couple the sound module to at least one headphone;
at least one headphone comprising an input connector configured to couple the at least one headphone to the output connector of the sound module; and
a computer-readable storage medium comprising executable instructions that cause a computer processor to perform steps in a self-administered hearing test, the steps comprising causing the sound module and the at least one headphone to generate pure tone stimuli calibrated to predetermined sound pressure levels without adjustment by a user administering the self-administered hearing test.

2. The kit of claim 1, wherein the sound module is an external sound module and the input connector comprises a universal serial bus connector configured to couple the external sound module to a computer.

3. The kit of claim 1, wherein the predetermined sound pressure levels are relative to Reference Equivalent Sound Pressure Levels specified in a standard issued by the American National Standards Institute.

4. The kit of claim 1, wherein the pure tone stimuli are generated within about +/−3 dB of the predetermined sound pressure levels for frequencies between about 400 Hz and about 4.5 kHz, and within about +/−6 dB of the predetermined sound pressure levels for frequencies between about 4.5 kHz and about 9 kHz.

5. The kit of claim 1, wherein the at least one headphone is selected from the group consisting of earphones, earbuds, stereophones, and headsets.

6. The kit of claim 1, wherein the steps in the self-administered hearing test further comprise receiving input signals from a user in response to the pure tone stimuli at predetermined frequencies and identifying user threshold levels for the predetermined frequencies based on the input signals.

7. The kit of claim 6, wherein the steps in the self-administered hearing test further comprise measuring one or more quality indicators related to an accuracy of the self-administered hearing test.

8. The kit of claim 6, wherein the steps in the self-administered hearing test further comprise communicating test result information based on the identified user threshold levels.

9. The kit of claim 8, wherein the test result information includes an interpretation of the identified user threshold levels.

10. The kit of claim 8, wherein the test result information includes a recommendation to the user based on the identified user threshold levels.

11. The kit of claim 8, wherein at least part of the test result information is communicated with an audio and/or video recording.

12. The kit of claim 11, wherein the steps in the self-administered hearing test further comprise setting a sound level for playback of the audio and/or video recording based on the identified user threshold levels.

13. A method for conducting a self-administered hearing test, comprising:
coupling a sound module to a computer;
coupling headphones to the sound module;
loading executable instructions onto the computer that program a processor of the computer to perform steps in a self-administered hearing test; and
conducting the self-administered hearing test with the computer to generate pure tone stimuli with the sound module and the headphones, the pure tone stimuli being calibrated to predetermined sound pressure levels without user adjustment.

14. The method of claim 13, wherein the sound module is an external sound module comprising a universal serial bus connector configured to couple the external sound module to a computer.

15. The method of claim 13, further comprising loading the executable instructions on the computer with a computer-readable storage medium.

16. The method of claim 13, further comprising loading the executable instructions on the computer by downloading the executable instructions over a network from a remote server.

17. The method of claim 13, wherein the pure tone stimuli are generated within about +/−3 dB of the predetermined sound pressure levels for frequencies between about 400 Hz and about 4.5 kHz, and within about +/−6 dB of the predetermined sound pressure levels for frequencies between about 4.5 kHz and about 9 kHz.

18. The method of claim 13, wherein the predetermined sound pressure levels are Reference Equivalent Sound Pressure Levels specified in a standard issued by the American National Standards Institute.

19. The method of claim 13, wherein the steps in the self-administered hearing test comprise receiving input signals from a user in response to the pure tone stimuli at predetermined frequencies and identifying user threshold levels for the predetermined frequencies based on the input signals.

20. The method of claim 19, wherein the steps in the self-administered hearing test further comprise communicating test result information based on the identified user threshold levels with an audio and/or video recording, and further comprise setting a sound level for playback of the audio and/or video recording based on the identified user threshold levels.

21. A system for conducting a self-administered hearing test, comprising:
a computer having a processor;
at least one headphone; and
a sound module coupled between the computer and the at least one headphone, the sound module being external to the computer,
wherein the processor is programmed with executable instructions for conducting a hearing test, and
wherein upon receiving signals from the computer, the sound module and the at least one headphone produce pure tone stimuli calibrated to predetermined sound pressure levels without user adjustment.

22. The system of claim 21, wherein the pure tone stimuli are generated within about +/−3 dB of the predetermined sound pressure levels for frequencies between about 400 Hz and about 4.5 kHz, and within about +/−6 dB of the predetermined sound pressure levels for frequencies between about 4.5 kHz and about 9 kHz.
